(19) United States
(12) Patent Application Publication
(10) Pub. No.: US 2005/0118291 A1
(43) Pub. Date: Jun. 2, 2005

(54) Formulations and methods for treating breast cancer with Morinda citrifolia and methylsulfonymethane

(76) Inventors: Mian-Ying Wang, Rockford, IL (US); Claude Jarakae Jensen, Cedar Hills, UT (US); Chen Su, West Jordan, UT (US); Stephen P. Story, Alpine, UT (US)

Correspondence Address: KIRTON & McCONKIE, 1800 Eagle Gate Tower, 60 East South Temple, Salt Lake City, UT, 84111, US

(21) Appl. No.: 10/937,419

(22) Filed: Sep. 9, 2004

Related U.S. Application Data
(60) Provisional application No. 60/501,728, filed on Sep. 10, 2003.

(51) Int. Cl. A61K 35/78

(52) U.S. Cl. 424/769; 424/777

(57) ABSTRACT

The present invention advances prior art techniques, formulations, and methods for treating mammary breast cancer during its initial phases, as well as for preventing mammary breast cancer, by providing a safe, nutraceutical formulation comprising Morinda citrifolia, methylsulfonylmethane (MSM), and other ingredients.

### 1. RELATED APPLICATIONS

This application claims priority through U.S. patent application Ser. No. 10/639,833 filed Aug. 12, 2003, and U.S. Patent Application Ser. No. 60/403,154 filed Aug. 12, 2002.

### BACKGROUND

1. Field of the Invention

The present invention is related to the inhibition and treatment of breast cancer using a nutraceutical composition comprising a quantity of a processed Morinda citrifolia product.

2. Background of the Invention and Related Art

Breast cancer is the second leading cause of cancer death in women (following lung cancer). Even allowing for improvements in detection (i.e., the introduction of routine mammography), there has been a long-term gradual increase in the incidence of breast cancer since the early 1970s, but because of the more effective treatment afforded by such early detection, overall mortality began to decrease by the mid-1990s. Breast cancers can arise in the lobes or lobules (lobular carcinoma) or in the ducts (ductal carcinoma) of the breast. Lobular carcinoma often affects both breasts.

Epidemiological study has identified certain risk factors that increase the possibility that a woman will get breast cancer, although not all women with breast cancer have these traits, and many women with all of these traits do not develop the disease. Risk factors include age (the incidence of breast cancer is rare in women under 35—most cases occur in women over 60); a history of breast cancer in a close blood relative; and a history of breast cancer or benign proliferative breast disease. A high cumulative exposure to female sex hormones (estrogen and progesterone) appears to increase the risk of some breast cancers. Hormonally related risk factors include early menarch (before age 12), late menopause (after age 55), having no children or postponing childbirth, and obesity in women over 50. Many other possible associations are under study, such as those relating to postmenopausal estrogen replacement, alcohol and fat consumption, lack of exercise, and exposure to pesticides and other environmental chemicals.

Like all cancers, breast cancers result from changes in the structure or function of genes that are key to the regulation of cellular growth, differentiation, or repair. Acquired changes in a number of specific genes have been associated with the disease; these are changes that occur during a person's lifetime but are not inherited or passed on. About 5% of women with breast cancer have an inherited susceptibility to the disease, and most of these women have an inherited mutation in one of two genes. In 1994 it was discovered that women who inherit a mutated BRCA1 gene have an almost 85% chance of developing breast cancer and an increased chance of developing uterine cancer. BRCA1 normally acts to prevent tumors by repairing damage to the genetic material caused by oxidation, a chemical process that in the body occurs naturally during metabolism. Defective BRCA1 genes cannot repair this damage, allowing its effects to accumulate over time. Cells with oxidative damage to the genes that control their growth can proliferate, or become cancerous. The defective gene can be inherited from either parent, but appears to cause breast cancer only in women. Young women who get breast cancer often come from families that carry a BRCA1 mutation. BRCA1 mutations account for about half of known hereditary breast cancers. Another gene, named BRCA2, has also been identified. BRCA2 mutations have been associated with both female and rare male breast cancers. The two genes may also play a role in some ovarian cancers and sporadic (nonhereditary) breast cancer cases. Seeking a natural way to prevent human cancer is an urgent task for cancer prevention investigators.

Most women who get breast cancer, do not have a genetic risk factor. Therefore, environmental carcinogen exposure may play an important role in human breast cancer development. Based upon scientific evidence, most chemical carcinogens need activation by enzymes to be transformed to a form that readily binds to genetic DNA to form DNA-adducts. Carcinogen-DNA adduct formation is an important “DNA damage” marker that predicts the possibility of cancer development. Most scientists agree that carcinogen induced DNA adduct formation is an early critical step in the multiple stages of carcinogenesis. Carcinogen-DNA adducts can be repaired by body enzymes. The unrepaired adducts will be fixed after one cell cycle. The unrepaired, fixed DNA damage will be responsible for mutation and the consequent cancer development. Therefore, preventing carcinogen-DNA adduct formation is a key step for cancer prevention at the initiation step of carcinogenesis. A formulation capable of preventing and or blocking the formation of carcinogen induced DNA adducts, may prevent cancer at the initiation stage of multiple stage carcinogenesis.

Cigarette smoking has been implicated in the pathogenesis of emphysema, ischemic heart diseases, and cancer. A series of authoritative reports by the U.S. Public Health Service, and other international scientific organizations, has conclusively documented a causal relationship between cigarette smoking and cancer in men and women. There are forty-eight known chemical carcinogens among the four thousand compounds detected in cigarettes. Most recently, it was reported that two-hundred twenty seven possible carcinogens exist in cigarettes. It was estimated that some 1×10¹⁷ oxidant molecules are present in each puff of cigarette smoke. Free radicals are known to cause oxidative damage and consequent lipid peroxidation, which are involved in the pathogenesis of human diseases. The induction of lipid peroxidation largely results from free radical reactions with polyunsaturated fatty acids in biological membranes. The unsaturated bonds undergo authocatalytic or enzymatic processing to form harmful lipid hydroperoxides. The active lipid hydroperoxides may be quickly converted to aldehydes, such as malondialdehyde, and alkenals, such as 4-hydroxynonenal. All of these are very active in DNA binding and are responsible for major indigenous cell damage.

Accumulating evidence indicates that cyclooxygenase-2 (“COX-2”) inhibitors may be involved in breast, colon, and lung cancer development. Interest in cancer chemoprevention with COX-2 inhibitors has been stimulated by epidemiological observations that the use of aspirin and other non-steroidal inflammatory drugs (NSAIDs) is associated with the reduced incidence of colon and breast cancer. The main target of NSAID activity is the cyclooxygenase (COX) enzyme. Two isoforms of COX have been identified: COX-1, the constitutive isoform, and COX-2, the inducible form of the enzyme. COX-2 can undergo rapid induction in response to chemical carcinogens. It has been suggested that COX-2 overexpression may lead to increased angiogenesis and inflammatory reaction. Therefore, the inhibition of COX-2 might have a general cancer preventive effect via anti-inflammatory activity and decrease angiogenesis.

Some studies suggest that physical activity, good nutrition, and the administration of certain drugs may lower a woman's risk of getting this deadly disease. Although progress has been achieved in the treatment of breast cancer, an effective way to prevent or inhibit the development of breast cancer is not available. Therefore, seeking a natural way to prevent human breast cancer becomes a top priority for the scientists who work in this field.

### SUMMARY AND OBJECTS OF THE INVENTION

The present invention advances prior art techniques, formulations, and methods for treating breast cancer during its initial phases, as well as for preventing and inhibiting breast cancer, by providing a safe, nutraceutical formulation comprising Morinda citrifolia, methylsulfonylmethane (MSM), and other ingredients. The preferred exemplary embodiments of the present invention improve upon existing systems and methods, and can, in some instances, be used to overcome one or more problems associated or related to such existing systems and methods.

In accordance with the invention as embodied and broadly described herein, the present invention features a nutraceutical formulation for treating and inhibiting mammary breast cancer comprising processed Morinda citrifolia present in an amount between about 0.001 and 99.9 percent by weight. In one preferred exemplary embodiment, the nutraceutical formulation further comprises methylsulfonylmethane (MSM) present in an amount between about 0.001 and 99.9 percent by weight.

These and other features and advantages of the present invention will be set forth or will become more fully apparent in the description that follows and in the appended claims. The features and advantages may be realized and obtained by means of the instruments and combinations particularly pointed out in the appended claims. Furthermore, the features and advantages of the invention may be learned by the practice of the invention or will be obvious from the description, as set forth hereinafter.

### BRIEF DESCRIPTION OF THE DRAWINGS

The foregoing and other objects and features of the present invention will become more fully apparent from the accompanying figures when considered in conjunction with the following description and appended claims. Although the figures depict only typical embodiments of the invention, and are thus not deemed limiting of the invention scope, the accompanying figures help explain the invention in added detail.

FIG. 1 is a graphical representation of the effectiveness of certain Morinda citrifolia-containing compounds on the prevalence of tumors in accordance with the present invention.

FIG. 2 is a graphical representation of the preventative effects of Morinda citrifolia-containing compounds on mammary gland tumorigenesis induced by estrogen in female ACI rats in accordance with the present invention.

FIG. 3 is a graphical representation of the relative body weight of rats that have been implanted with estrogen to induce tumorigenesis, wherein certain rats have been treated with Morinda citrifolia-containing compounds to counteract the effects of estrogen-induced tumorigenesis in accordance with the present invention.

FIG. 4 is a graphical representation of the relative size of tumors in rats treated with various compounds.

FIG. 5 is a graphical representation of the conversion of Dimethyl Sulfoxide to Methyl Sulfonyl Methane in accordance with certain embodiments of the present invention.

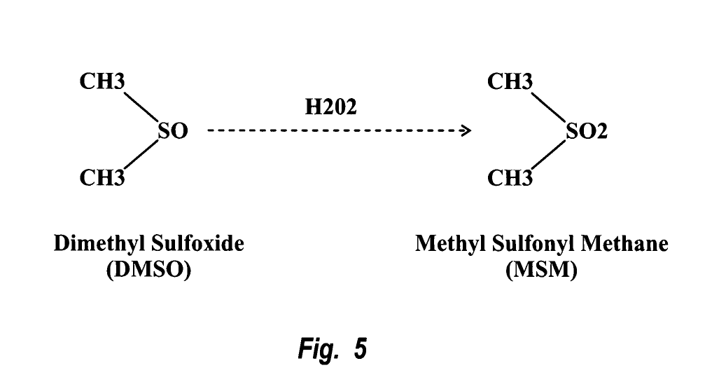

### DETAILED DESCRIPTION OF THE PREFERRED EMBODIMENTS

The present invention relates to techniques, formulations, and methods for treating breast cancer during its initial phases, as well as for inhibiting breast cancer, by providing a safe, nutraceutical formulation comprising Morinda citrifolia, methylsulfonylmethane (MSM), and other ingredients. The preferred exemplary embodiments of the present invention improve upon existing systems and methods, and can be used to overcome one or more problems associated or related to such existing systems and methods.

The following disclosure of the present invention is grouped into three subheadings, namely “General Discussion of Morinda citrifolia and the Methods Used to Produce Processed Morinda citrifolia Products,” “Formulations and Methods of Administration” and “Preventing, Inhibiting and Treating Breast Cancer.” The utilization of the subheadings is for convenience of the reader only and is not to be construed as limiting in any sense.

It will be readily understood that the elements of the present invention, as generally described and illustrated in the figures herein, could be combined and used in a wide variety of different formulations and methods. Thus, the following more detailed description of the embodiments of the system and method of the present invention is not intended to limit the scope of the invention, as claimed, but is merely representative of the presently preferred embodiments of the invention.

1. General Discussion of Morinda citrifolia and the Methods Used to Produce Processed Morinda citrifolia Products

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the nutraceutical used to prevent, inhibit and/or treat breast cancer. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 120 days. Most fruit containers are held for 7 to 14 days before processing. The containers can optionally be stored under refrigerated conditions or ambient/room temperature conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 0.01 micron up to 2000 microns, more preferably less than 500 microns, a filter press, reverse osmosis filtration, and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include: (from the leaves): alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; (from the flowers): acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; (from the fruit): acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; (from the roots): anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; (from the root bark): alizarin, chlororubin, glycosides (pentose, hexose), morindadiol, morindanigrine, morindine, morindone, resinous matter, rubiadin monomethyl ether, and soranjidiol; (from the wood): anthragallol-2,3-dimethylether; (from the tissue culture): damnacanthal, lucidin, lucidin-3-primeveroside, and morindone-6beta-primeveroside; (from the plant): alizarin, alizarin-alpha-methyl ether, anthraquinones, asperuloside, hexanoic acid, morindadiol, morindone, morindogenin, octanoic acid, and ursolic acid. The present invention contemplates utilizing all parts of the M citrifolia plant alone, in combination with each other or in combination with other ingredients. The above listed portions of the M citrifolia plant is not an exhaustive list of parts of the plant to be used but are merely exemplary. Thus, while some of the parts of the M citrifolia plant are not mentioned above (e.g., seed from the fruit, the pericarp of the fruit, the bark or the plant) the present invention contemplates the use of all of the parts of the plant.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

The compositions containing Morinda citrifolia may be in a form suitable for oral use, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients, which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

2. Formulations and Methods of Administration

The present invention provides nutraceutical formulations and methods for preventing, inhibiting and treating breast cancer with a Morinda citrifolia-based nutraceutical formulation without any significant tendency to cause side effects. The Morinda citrifolia is incorporated into various carriers or nutraceutical compositions suitable for in vivo treatment of a patient. For instance, the processed Morinda citrifolia may be ingested, introduced through an intravenous injection or feeding, or otherwise internalized as is appropriate and directed.

Methylsulfonymethane (“MSM”) is a sulfur donor compound occurring in nature and has been found in plants, milk, and urine of bovines and humans. MSM is a normal oxidative product of dimethylsulfoxide (DMSO). Sulfur is the sixth most abundant macro-mineral in breast milk and the third most abundant mineral in the human body based upon the percentage of total body weight and is an essential element for the structure of every living cell. MSM possesses a broad range of health benefits including analgesic, anti-inflammatory, anti-allergy, while enhancing immune function by providing nutritionally essential organic sulfur and methyl groups. A scientific study has reported that ³⁵S-labelled MSM was incorporated into essential sulfur-containing amino acids such as in methionine and cysteine of guinea pig serum protein; thus MSM may provide a source of sulfur for essential sulfur-containing amino acids in animals and humans.

In one exemplary embodiment, the nutraceutical composition of the present invention comprises one or more of a processed Morinda citrifolia product present in an amount by weight between about 0.01 and 100 percent by weight, and preferably between 0.01 and 95 percent by weight combined with methylsulfonylmethane (“MSM”), present in an amount between about 0.001 and 99.9 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product is an active ingredient or contains one or more active ingredients, such as Quercetin and Rutin, and others, for effectuating the prevention, inhibition and treatment of breast cancer. The effects of the processed Morinda citrifolia product are synergistically enhanced by the presence of methylsulfonylmethane (“MSM”) in formulation. One embodiment of the present invention comprises a processed Morinda citrifolia product combined in formulation with MSM that prevents, inhibits and or treats breast cancer. Active ingredients may be extracted out of various parts of the Morinda citrifolia plants using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent.

The processed Morinda citrifolia product may be formulated with various other ingredients to produce various compositions, such as a nutraceutical composition, an internal composition, or others. The ingredients to be utilized in a nutraceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the nutraceutical composition will most likely be consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically administered formulations may also comprise any known in the art.

In one exemplary embodiment, the present invention further features a method of administering a nutraceutical composition to a mammal for the prevention, inhibition or treatment of breast cancer. The method comprises the steps of (a) formulating a nutraceutical composition comprising in part a processed Morinda citrifolia product present in an amount between about 0.01 and 95 percent by weight and methylsulfonylmethane (“MSM”), present in an amount between about 0.001 and 80 percent by weight, wherein the composition also comprises a carrier, such as water or purified water, and other natural or artificial ingredients; (b) administering the nutraceutical composition into the body such that the processed Morinda citrifolia product is sufficiently internalized; (c) repeating the above steps as often as necessary to provide an effective amount of the processed Morinda citrifolia product.

The step of administering the nutraceutical composition into the body comprises ingesting the composition orally through one of several means. Specifically, the nutraceutical composition may be formulated as a liquid, gel, solid, or some other type that would allow the composition to be quickly and conveniently digested. Once sufficiently internalized, the administered nutraceutical composition may then begin to act prevent, inhibit or treat breast cancer in the subject. In addition, the step of administering the nutraceutical composition may include injecting the composition into the body using an intravenous pump.

In one exemplary embodiment, the nutraceutical composition is administered by taking between two ounces, of the nutraceutical composition every two hours each day, or at least twice a day. The nutraceutical composition is to be taken on an empty stomach, meaning at a period of time at least two hours prior to consumption of any food or drink. Of course, one ordinarily skilled in the art will recognize that the amount of composition and frequency of use may vary from individual to individual. It is contemplated that a person may ingest less than one-half ounce, or more than ten ounces of the nutraceutical composition claimed in the present invention. Thus, the present invention contemplates the administration of one ounce, two ounces, three ounces or any volume of the formulations necessary to achieve the desired result including more than ten ounces per administration.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

Formulation One

| | |
|---|---|
| Morinda citrifolia puree juice or fruit juice | 100% |

Formulation Two

| | |
|---|---|
| Morinda citrifolia fruit juice | 50-99.99% |
| water | 0.1-50% |

Formulation Three

| | |
|---|---|
| Morinda citrifolia fruit juice | 50-99.99% |
| non-Morinda citrifolia-based fruit juices | 0.1-50% |

Formulation Four

| | |
|---|---|
| Morinda citrifolia fruit juice | 50-90% |
| water | 0.1-50% |
| non-Morinda citrifolia-based fruit juices | 0.1-30% |

Formulation Five

| | |
|---|---|
| Morinda citrifolia puree juice | 50-99.9% |
| water | 0.1-50% |

Formulation Six

| | |
|---|---|
| Morinda citrifolia puree juice | 50-99.9% |
| non-Morinda citrifolia-based fruit juices | 0.1-50% |

Formulation Seven

| | |
|---|---|
| Morinda citrifolia puree juice | 50-90% |
| water | 0.1-50% |
| non-Morinda citrifolia-based fruit juices | 0.1-30% |

Formulation Eight

| | |
|---|---|
| Morinda citrifolia dietary fiber | 0.1-50% |
| water | 1-99.9% |
| non-Morinda citrifolia-based fruit juices | 1-99.9% |

Formulation Nine

| | |
|---|---|
| Morinda citrifolia dietary fiber | 0.1-50% |
| water | 1-99.9% |
| Morinda citrifolia fruit juice or puree juice | 1-99.9% |

Formulation Ten

| | |
|---|---|
| Morinda citrifolia oil | 0.1-50% |
| carrier medium | 70-99.9% |
| other ingredients | 1-95% |

Formulation Eleven

| | |
|---|---|
| Morinda citrifolia product | 10-80% |
| carrier medium | 20-90% |

Formulation Twelve

| | |
|---|---|
| Morinda citrifolia product | 5-80% |
| carrier medium | 20-95% |

Formulation Thirteen

| | |
|---|---|
| Morinda citrifolia oil or oil extract | 0.1-50% |
| carrier medium | 20-90% |

Formulation Fourteen

| | |
|---|---|
| Morinda citrifolia puree juice or fruit Juice | 0.1-80% |
| Morinda citrifolia oil | 0.1-50% |
| carrier medium | 20-90% |

Formulation Fifteen

| | |
|---|---|
| Morinda citrifolia puree juice concentrate | 100% |
| or fruit juice concentrate | |

Formulation Sixteen

| | |
|---|---|
| Morinda citrifolia fruit juice concentrate | 50-99.99% |
| or puree juice concentrate | |
| water | 0.1-50% |

Formulation Seventeen

| | |
|---|---|
| MSM | 0-80% |
| Morinda citrifolia puree juice or fruit juice | 20-100% |

Formulation Eighteen

| | |
|---|---|
| MSM | 0.1-49.9% |
| Morinda citrifolia fruit juice | 50-99.8% |
| water | 0.1-50% |

Formulation Nineteen

| | |
|---|---|
| MSM | 0.1-49.9% |
| Morinda citrifolia fruit juice | 50-99.99% |
| non-Morinda citrifolia-based fruit juices | 0.1-50% |

Formulation Twenty

| | |
|---|---|
| MSM | 0.1-49.8% |
| Morinda citrifolia fruit juice | 50-90% |
| water | 0.1-50% |
| non-Morinda citrifolia-based fruit juices | 0.1-30% |

In one preferred method, a person wanting to prevent, inhibit or treat breast cancer as described above takes, or is administered, at least one ounce of Formulation One in the morning on an empty stomach, and at least one ounce at night on an empty stomach, just prior to retiring to bed. In one example, which is not meant to be limiting in any way, the beneficial Morinda Citrifolia is processed into Tahitian Noni® juice manufactured by Morinda , Incorporated of Orem, Utah.

According to the present invention, these particular methods of introducing an internal composition may comprise any method of actually introducing the internal composition to the subject for the purpose of preventing, inhibiting, or treating breast cancer. Although the particular methods are many, the present invention recognizes that the internal composition may be introduced intravenously, transdermally, orally, or systemically. No matter what method is employed, it is important to regulate the amount of active ingredient that the subject is exposed to so that the appropriate anti-cancer objectives are accomplished.

The carrier medium may comprise any ingredient capable of being introduced into the body of a mammal, and that is capable of providing the carrying medium to the processed Morinda citrifolia product and MSM. Specific carrier mediums formulations are well known in the art and are not described in detail herein. The purpose of the carrier medium is as stated, to provide a means to embody the processed Morinda citrifolia product and MSM within the internal composition that is capable of being introduced into the body of the subject to be treated.

The following examples set forth and present the effects of preventing, inhibiting, or treating breast cancer with Morinda citrifolia. These examples are not intended to be limiting in any way, but are merely illustrative of the benefits and advantages of utilizing Morinda citrifolia to prevent, inhibit and treat breast cancer.

3. Preventing, Inhibiting and Treating Breast Cancer

The following examples set forth and present the effects of Morinda citrifolia on carcinogenic cells. These examples are not intended to be limiting in any way, but are merely illustrative of the beneficial, advantageous, and remedial effects of Morinda citrifolia on carcinogenic cells, including carcinogenic cells located in mammalian mammaries. Other non-limiting examples of the present invention are described below.

The present invention describes formulations and methods for preventing, inhibiting and treating breast cancer during the initiation stages of breast cancer using a nutraceutical composition formulated with a quantity of a processed Morinda citrifolia product. The present invention relates to the synergistic cancer prevention effects of Morinda citrifolia, and a Morinda citrifolia and methylsulfonylmethane combination regimen at the initiation stage of breast cancer.

### EXAMPLE ONE

### Formula and Method of Administering Morinda citrifolia and Methylsulfonylmethane

In the present example, a patient with breast cancer desires to treat the condition with a nonprescription, over-the-counter preparation. To treat the cancer, the individual consumes an identified prescribed amount of a food product composition containing processed Morinda citrifolia fruit juice and methylsulfonylmethane (“MSM”). The person intermittently consumes the food product containing the processed Morinda citrifolia fruit juice and MSM until the carcinogenic cells are inhibited, blocked, and/or destroyed.

In another embodiment of the present invention a person interested in preventing or inhibiting the development of carcinogenic tissues may consume a food product compositions containing processed Morinda citrifolia fruit juice and MSM. The person intermittently consumes the food product containing the processed Morinda citrifolia fruit juice and MSM for an indefinite period to continually prevent or inhibit the development of carcinogenic tissues.

### EXAMPLE TWO

### Prevention, Inhbition and Treatment of Cancer with Processed Morinda citrifolia Products and Methylsulfonylmethane

Processed Morinda citrifolia products possess cancer preventive effects at the initiation stage of chemical carcinogenesis. This hypothesis was examined using two carcinogenic animal models, and one human clinical study of a group of current smokers. The animal models included the following: the DMBA-induced mammary gland tumorigenesis model, and an acute liver injury model induced by a liver carcinogen, carbon tetrachloride (CC14). These are classical extrinsic carcinogenic models. DMBA induced DNA adduct formation, in addition to histological examination by light and electron microscopy, was chosen as a sensitive biomarker to evaluate the preventive effect of processed Morinda citrifolia products at the initiation stage of multiple step carcinogenesis. In the mammary gland carcinogenic model, the focus was on the pathogenic changes after DMBA administration, to monitor the mechanisms of carcinogenesis and DMBA DNA-adduct formation in mammary tissue. In the acute liver injury model, the histopathological changes of liver tissue and the superoxide anion free radicals (SAR) and lipid hydroperoxide (LPO) levels after CC14 administration were the focus.

Carcinogen DMBA DNA-adduct formation was used as a marker to examine whether processed Morinda citrifolia products were able to prevent carcinogen induced DNA damage. The cancer preventive effects of processed Morinda citrifolia products at the initiation stage of mammary breast carcinogenesis, induced by DMBA, was examined in female Sprague-Dawley (SD) rats. The experiment was started at the 35^{th} postnatal day with water in an age-matched control group, a DMBA group, and a 5% processed Morinda citrifolia products group. DMBA (25 mg/kg) was administrated by mouth at the 50^{th} postnatal day in the DMBA and processed Morinda citrifolia products groups. processed Morinda citrifolia products were continuously supplied for an additional 90 days after DMBA administration. All the animals were sacrificed at the 8^{th} month after DMBA administration to examine the pathological changes in the mammary glands by light microscopy. Compared to controls, the DMBA treated group showed a variety of lesions, including epithelial hyperplasias (12.5%), benign tumors (25%), and in-situ carcinomas (25%). No benign tumors or carcinomas were found in the processed Morinda citrifolia products group, which showed normal histology or mild hyperplasia. These results indicate that processed Morinda citrifolia products may prevent breast cancer at the initiation stage of chemical carcinogenesis.

In another study, the preventive effect of processed Morinda citrifolia products on carbon tetrachloride (CC14)-induced liver injury in female SD rates was examined by light microscopy (LM) and electron microscopy (EM) examination. Liver sections in placebo and processed Morinda citrifolia products groups demonstrated normal lobular architecture and normal ultrastructure at the LM level. Liver sections in the placebo+CC14 group showed acute liver damage at the LM level: which includes focal vacuolated, lipid-containing or necrotic hepatocytes surrounding central veins and focal inflammatory cells scattered throughout the lobule. There was a significant decrease in the number of swollen, lipid containing, and apoptotic hepatocytes in the processed Morinda citrifolia products+CC14 group, compared to the placebo+CC14 group. At the EM level, glycogen depletion and lipid droplets in the cell plasma were observed in both CC14 treated groups. Swollen mitochondria, disorganization of rough endoplasmic reticulum (RER) with loss of ribosomes, and abundant focal areas of smooth endoplasmic reticulum (SER) were scattered throughout the cytoplasm. Interestingly, Golgi complexes in placebo+CC14 group contain small low-density vesicles. Golgi complexes in the processed Morinda citrifolia products+CC14 group contain large vesicles with increased electron density, and Golgi cistemal stacks were well developed. Those in the placebo+CC14 group were often swollen and diminished.

Possible mechanism of the cancer preventative effect of processed Morinda citrifolia products were studied. Female SD rats were divided into two groups of six each. The control group was given regular drinking water and rat show, ad libitum. The processed Morinda citrifolia products group was given 10% processed Morinda citrifolia products in drinking water and rat chow, ad libitum. One week later, three animals from each group received intragastrically 25 mg/kg of DMBA containing 5% dimethysulfoxide in corn oil. All animals were sacrificed 24 h later. DNA was isolated from liver, lung, heart, and kidney. The DNA adducts were analyzed by P-postlabeling technique. After one week of consumption, the processed Morinda citrifolia products group showed a reduction in both the number and level of DMBA-DNA adducts from each of the four organs studied. The quantitative estimate after radioactive counting indicated that processed Morinda citrifolia products reduced the amount of DNA adduct formation by 80% in kidney, 42% in liver, 41% in lung, and 26% in heart. Even more dramatic experimental results were obtained using male C57 BL-6 mice. Processed Morinda citrifolia products were able to reduce the formation of DMBA-DNA adducts by 90% in kidney, 70% in liver, 60% in heart, and 50% in lung. This is the first finding of the cancer preventive effect at the initiation stage of carcinogenesis by processed Morinda citrifolia products. This data indicates that processed Morinda citrifolia products may prevent cancer at the initiation stage of carcinogenesis.

In order to explore the mechanisms of the cancer preventive effect of processed Morinda citrifolia products, the antioxidant activity was examined. The study was designed to measure how well processed Morinda citrifolia products scavenged superoxide anion radicals (SAR) and quenched lipid peroxides (LPO) by TNB assay and LMB assay, respectively. SAR scavenging activity was examined in vitro by tetrazolium nitroblue (TNB) assay. In TNB assay, SAR reduces TNB into formazan blue, which absorbs at 602 mm. A SAR scavenger, such as processed Morinda citrifolia products, reduces the absorbency by reacting with SAR. In this assay, a standard curve is produced when SAR are generated from NADH under aerobic conditions, with phenazine methosulfate as a catalyst. In LMB assay, LPO oxidizes leucomethylene to methylene blue in the presence of hemoglobin. The resultant blue color can be quantified spectrophotometrically at 660 nm.

In vitro processed Morinda citrifolia products showed a dose-dependent inhibition of both LPO and SAR. The SAR scavenging activity of processed Morinda citrifolia products was compared to that of three known antioxidants: Vitamin C, grape seed powder, and Pycnogenol at the daily dose per serving level recommended by US RDA's or manufacturer's recommendations. Under the experimental conditions, the SAR scavenging activity of processed Morinda citrifolia products was shown to be 2.8 times that of vitamin C, 1.4 times that of Pycnogenol, and 1.1 times that of grape seed powder. Therefore, processed Morinda citrifolia products has a great potential to scavenge reactive oxygen free radicals.

Carbon tetrachloride is a liver carcinogen and lipid hydroperoxidation inducer. To further confirm the antioxidant activity of processed Morinda citrifolia products in vivo, a carbon tetrachloride induced liver injury model in female SD rats was selected. Ten percent of processed Morinda citrifolia product in drinking water for 12 days was able to reduce the liver LPO and SAR levels to 20% and 50% of that observed in the placebo group 3 hours after CC14 administration. In conclusion, processed Morinda citrifolia products may protect liver from an extrinsic carcinogenic CC14 exposure.

Antioxidants in processed Morinda citrifolia products may protect individuals from cigarette smoke by scavenging oxygen free radicals and quenching lipid peroxides. In order to examine this hypothesis, a one-month double blinded, randomized, and placebo-controlled clinical trial was designed to test the protective effect of processed Morinda citrifolia products on plasma SAR and LPO in current smokers. The subjects were supplemented daily with two ounces of processed Morinda citrifolia products (n=38) or placebo (n=30), twice a day for 30 days. The plasma SAR and LPO levels were determined before and after trial by TNB and LPO assay, respectively. There was no effect observed on plasma SAR (0.23±0.15 versus 0.21±0.17 μmol/mL) and LPO (0.58±0.22 versus 0.59±0.21 μmol/mL, P<0.05), respectively. These results indicate that processed Morinda citrifolia products may protect individuals from oxidative damage induced by tobacco smoke. Smoking specific, lipid peroxides and the related decomposed products such as malondialdehyde, induced DNA adducts will be analyzed soon.

The data from the in vitro study, CC14-induced liver injury model of female SD rats, and current smokers indicate that processed Morinda citrifolia products is a strong antioxidant which can scavenge reactive oxygen free radicals and quench lipid hydroperoxides, therefore reducing the cancer risk.

### EXAMPLE THREE

### Anti-Inflammatory Action and Selective COX-2 Inhibition by Processed Morinda citrifolia Products

In this study, the selectivity of COX-2 inhibition of processed Morinda citrifolia products versus COX-1 in vitro was investigated. The inhibitions of processed Morinda citrifolia products on COX-2 and COX-1 activities were compared with that of the traditional NSAIDs such as Aspirin, Indomethacin, and a known selective COX-2 inhibitor, Celebrex. The COX-1 and COX-2 activities were determined based upon the PGE2 levels generated during the incubations of human platelets with tested compounds and/or vehicle by the Amersham ELA assay. The IC of processed Morinda citrifolia products, Aspirin, Indomethacin, and Celebrex on COX-1 are 5%, 4.55 μmol/L, 0.01 μmol/L, and 1.4 μmol/L, respectively, and that for COX-2 are 3.8%, 595 μmol/L, 0.4 μmol/L, and 0.47 μmol/L respectively. The data was converted into a ratio of IC₅₀ COX-2/COX-1. It was 0.76 for processed Morinda citrifolia products, 119 for Aspirin, 40 for Indomethacin, and 0.34 for Celebrex. These results show that the selectivity of COX-2 inhibition of processed Morinda citrifolia products is comparable with that of Celebrex. The discovery of the selective COX-2 inhibition of processed Morinda citrifolia products is very significant since processed Morinda citrifolia products is a natural fruit juice without side effects. This is the first scientific evidence for a strong anti-inflammatory activity in processed Morinda citrifolia products, which may also be one mechanism of cancer prevention.

The anti-inflammatory activity of processed Morinda citrifolia products was observed in an acute liver injury model in female SD rats induced by CC14. A decrease in inflammatory foci and lymphocytes surrounding central vein areas were observed at 6 hours post CC14 administration in animals pretreated with 10% processed Morinda citrifolia products for twelve days in drinking water compared with the CC 14 group without processed Morinda citrifolia products.

### EXAMPLE FOUR

### Effect of Morinda citrifolia—Containing Compounds on E2-Induced Mammary Tumors

Sixty five-week old female rats were divided into four groups of fifteen rats each and placed on regular diets. Another eight female rats served as age-matched controls. One group of experimental rats was given 5% placebo in drinking water, the second experimental group was given 5% Morinda citrifolia juice in drinking water, the third experimental group was given 5% Methylsulfonylmethane (“MSM”) in drinking water, and the fourth experimental group was given a combination of 5% Morinda citrifolia juice and 5% MSM in drinking water. Two weeks later, all animals were implanted subcutaneously with a 25 mg pellet containing 22.5 mg of 17β-estradiol (E2) mixed with 2.5 mg cholesterol. Each experimental group was provided with its respective formulation for ninety days following estrogen (E2) implantation. The age-matched control animals received a 25 mg cholesterol pellet implant.

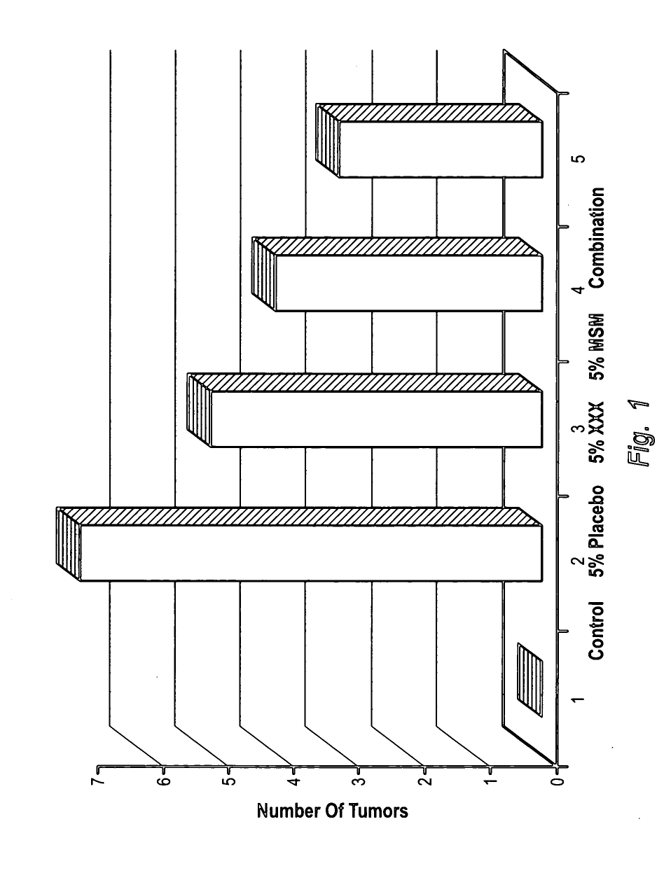

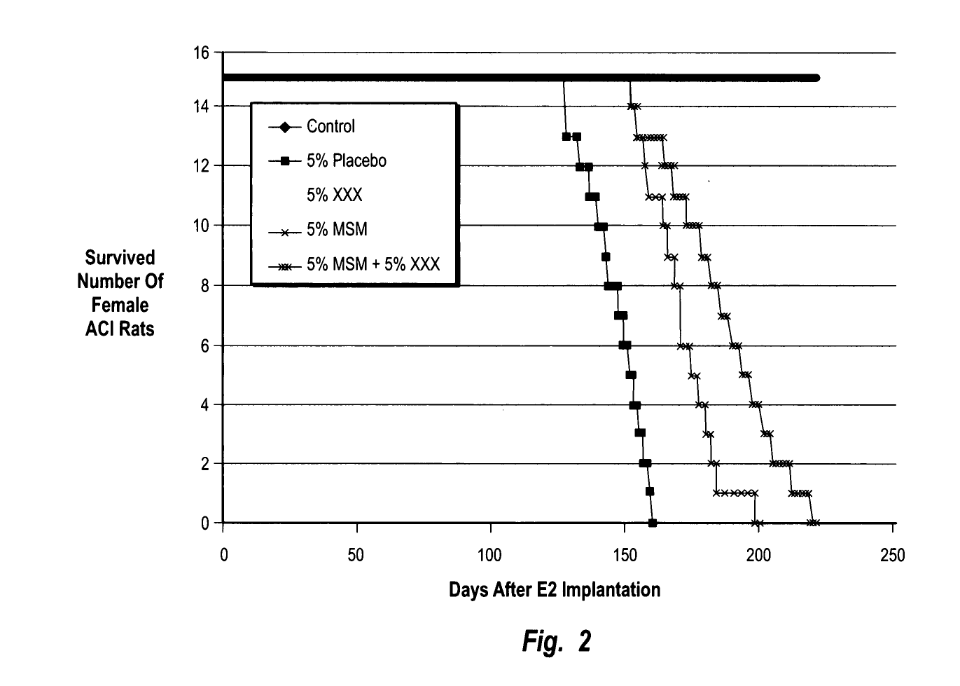

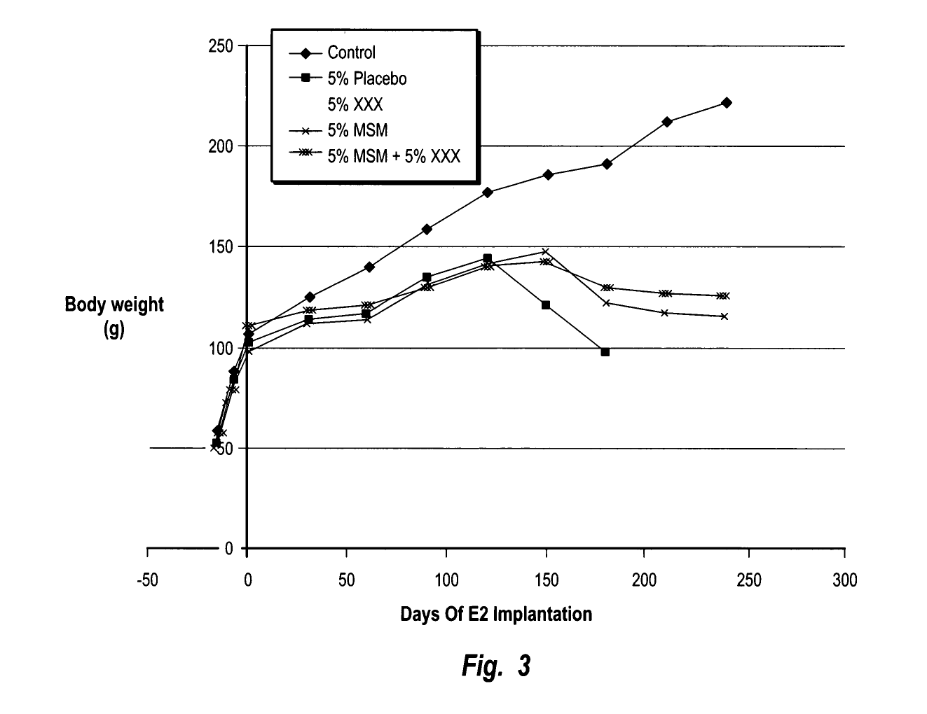

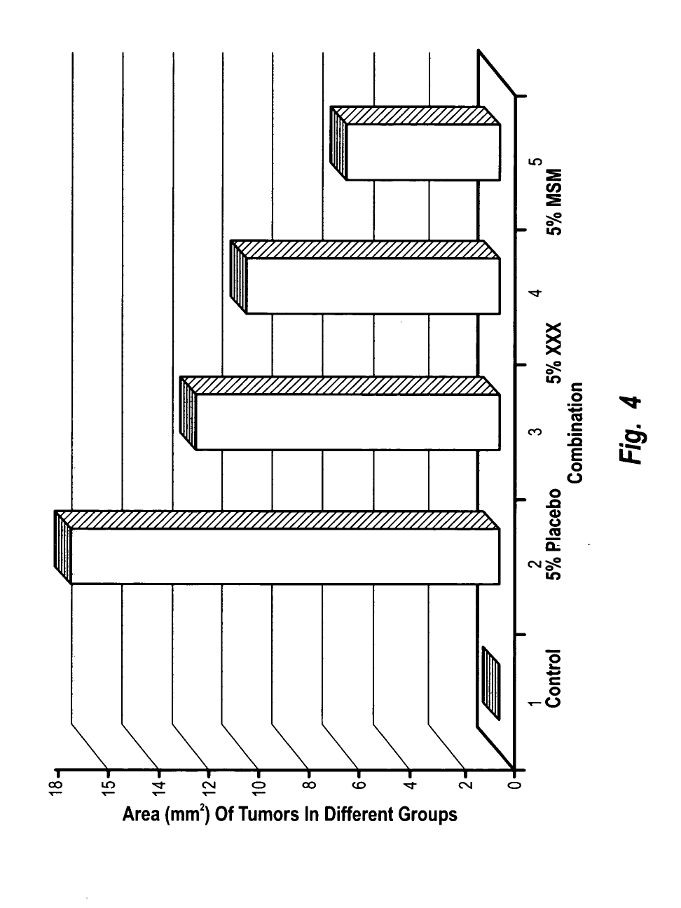

As seen in FIGS. 1-4, the animals in the placebo group had a significant body weight loss when compared to the cholesterol control group. The animals in the Morinda citrifolia group or MSM group had slight body weight loss. None of the rats that received the pellets composed of cholesterol exhibited mammary tumors. All rats with an E2 implant in the placebo group had mammary gland tumors. One hundred percent (100%) of the rats in this group had three to seven tumors. Seventy-one percent (71%) of the rats in the Morinda citrifolia group had two to five tumors. Fifty-seven percent (57%) of the rats in the MSM group had one to four tumors. Forty-three percent (43%) of the rats in the combination group had zero to three tumors.

The average tumor area in the placebo group, Morinda citrifolia group, MSM group, and combination group at 180 days after E2 implantation were 17, 12, 10 and 6 mm², respectively. The survival rates of the control, placebo, Morinda citrifolia, MSM and combination groups one hundred and sixty days after E2 implantation were 100%, 0%, 47%, 73%, and 87%, respectively. The survival rates of each group at one hundred eighty days after E2 implantation was 100%, 0%, 0%, 20% and 60%, respectively. At two hundred days, the survival rates of each group were 100%, 0%, 0%, 0%, and 27%, respectively.

### EXAMPLE FIVE

### Synergistic Effects of Morinda citrifolia and Methylsulfonylmethane

Processed Morinda citrifolia products, namely fruit juice, and methylsulfonylmethane (“MSM”) possess cancer inhibitive effect at the initiation stage of DMBA-induced mammary carcinogenesis in female SD rats. The combination of Morinda citrifolia and MSM have a synergistic cancer inhibitive effect at the initiation stage of mammary carcinogenesis induced by DMBA in female SD rats.

In research supporting the present invention, the cancer preventive effects of Morinda citrifolia, MSM, and their combination at the initiation stage of multiple stage chemical carcinogenesis was investigated on a mammary breast carcinogenic animal model induced by DMBA in female SD rats.

In the experiment, seventy-five female SD rats were divided into five groups: age-matched control, DMBA, 5% Morinda citrifolia+DMBA, 5% MSM+DMBA, and 5% Morinda citrifolia+5% MSM+DMBA groups. The experiment was started at the 35th postnatal day with water being given to the age-matched control group and the DMBA group, while 5% Morinda citrifolia, 5% MSM, and 5% Morinda citrifolia+5% MSM was supplied to the Morinda citrifolia, MSM, and Morinda citrifolia+MSM+DMBA groups. DMBA (25 mg/kg) was administered by mouth at the 50th postnatal day in the DMBA, Morinda citrifolia+DMBA, MSM+DMBA, and Morinda citrifolia+MSM+DMBA groups. Morinda citrifolia, MSM, Morinda citrifolia+MSM were continuously supplied for an additional 90 days after DMBA administration. All animals were sacrificed at the 9th month after DMBA treatment to examine the pathological changes in the mammary glands by light microscopy. Compared to the age-matched control group, the DMBA treated group showed a variety of lesions, including epithelial hyperplasia (12.5%), benign tumors (25%), and carcinomas in-situ (25%). No benign tumor or carcinoma was observed in the age-matched control, Morinda citrifolia, and MSM groups, which only showed normal histology or mild hyperplasia (10% in the age-matched control group, 60% in the Morinda citrifolia group, 75% in the MSM group). In the Morinda citrifolia and MSM combination group, mild hyperplasia was significantly decreased to 37.5%, which was 50% lower than that of the 5% MSM group, and 37.5% lower than that of the 5% Morinda citrifolia group (p<0.05). Our results indicate that a Morinda citrifolia and MSM combination regimen possesses a synergistic cancer preventive effect at the initiation stage of mammary carcinogensis in the DMBA-induced mammary tumor model. Therefore, this synergistic combination regimen may contribute to human breast cancer prevention at the initiation stage of carcinogenesis. This is the first finding to reveal this synergistic effect in breast cancer prevention. The advantages of this specific combination provide an effective, natural, safe, economic method to prevent breast cancer and no toxicity for long-term use. It is worth to point out that this combination regimen has been supplied only 90 days after carcinogen administration. Whether this combination regimen is able to prevent breast mammary tumor completely with a long-term treatment needs further study, and it may be a possible strategy for cancer prevention. The combination regimen may reverse the initiated cell back to normal.

While illustrative embodiments of the invention have been described herein, the present invention is not limited to the various preferred embodiments described herein, but includes any and all embodiments having modifications, omissions, combinations (e.g., of aspects across various embodiments), adaptations and/or alterations as would be appreciated by those in the art based on the present disclosure. The limitations in the claims are to be interpreted broadly based the language employed in the claims and not limited to examples described in the present specification or during the prosecution of the application, which examples are to be construed as non-exclusive. For example, in the present disclosure, the term “preferably” is non-exclusive and means “preferably, but not limited to.”

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

1. A nutraceutical composition for inhibiting and treating breast cancer, said nutraceutical composition comprising:
one of a processed Morinda citrifolia product and an extract present in an amount between about 0.001 and 99.9 percent by weight, and
methylsulfonylmethane present in an amount between about 0.001 and 99.9 percent by weight.

2. The nutraceutical formulation of claim 1, wherein said processed Morinda citrifolia product is comprised of at least one of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

3. The nutraceutical formulation of claim 1, wherein said Morinda citrifolia product is used with a carrier medium.

4. The nutraceutical formulation of claim 1, wherein said composition is administered by process comprising one or more of the following methods: orally, transdermally, injection, intravenously, topically or systemically.

5. The nutraceutical formulation of claim 1, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

6. The nutraceutical formulation of claim 1, wherein said composition is comprised of 0.1-49.9% methylsulfonylmethane by weight, 50-90% Morinda citrifolia fruit juice by weight, and 0.1-30% non-Morinda citrifolia based fruit juices by weight.

7. The nutraceutical formulation of claim 1, additionally comprising 0.1-50% water by weight.

8. A nutraceutical composition for preventing and inhibiting DNA adduct formation, said nutraceutical composition comprising:
processed Morinda citrifolia product or extract present in an amount between about 0.001 and 99.9 percent by weight, and
methylsulfonylmethane present in an amount between about 0.001 and 80 percent by weight.

9. The nutraceutical formulation of claim 8, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

10. The nutraceutical formulation of claim 8, wherein said Morinda citrifolia product is used with a carrier medium.

11. The nutraceutical formulation of claim 8, wherein said composition is administered by process comprising one or more of the following methods: orally, transdermally, injection, intravenously, topically or systemically.

12. The nutraceutical formulation of claim 8, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

13. The nutraceutical formulation of claim 8, wherein said composition is comprised of 0.1-49.9% methylsulfonylmethane by weight, 50-90% Morinda citrifolia fruit juice by weight, and 0.1-30% non-Morinda citrifolia based fruit juices by weight.

14. The nutraceutical formulation of claim 8, additionally comprising 0.1-50% water by weight.

15. A nutraceutical composition for chemically preventing and inhibiting carcinogenesis at the initiation stage by chemically blocking carcinogens, scavenging free radicals, quenching lipid peroxides, and inhibiting COX-2, said nutraceutical composition comprising:
processed Morinda citrifolia product or extract present in an amount between about 0.001 and 99.9 percent by weight, and
methylsulfonylmethane present in an amount between about 0.001 and 80 percent by weight.

16. The nutraceutical formulation of claim 15, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

17. The nutraceutical formulation of claim 15, wherein said Morinda citrifolia product is used with a carrier medium.

18. The nutraceutical formulation of claim 15, wherein said composition is administered by process comprising one or more of the following methods: orally, transdermally, injection, intravenously, topically or administered systemically.

19. The nutraceutical formulation of claim 15, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

20. The nutraceutical formulation of claim 15, wherein said composition is comprised of 0.1-49.9% methylsulfonylmethane by weight, 50-90% Morinda citrifolia fruit juice by weight, and 0.1-30% non-Morinda citrifolia based fruit juices by weight.

21. The nutraceutical formulation of claim 1, additionally comprising 0.1-50% water by weight.

22. A method for treating, inhibiting, and preventing mammary breast cancer comprising:
administering a nutraceutical composition to a patient, wherein said nutraceutical composition comprises processed Morinda citrifolia product or extract present in an amount between about 0.001 and 100 percent by weight, and methylsulfonylmethane present in an amount between about 0.001 and 99 percent by weight.

23. A method as in claim 22, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

24. A method as in claim 22, wherein said Morinda citrifolia product is used with a carrier medium.

25. A method as in claim 22, wherein said composition is administered by process comprising one or more of the following methods: orally, transdermally, injection, intravenously, topically or administered systemically.

26. A method as in claim 22, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

27. A method as in claim 22, wherein said composition is comprised of 0.1-49.9% methylsulfonylmethane by weight, 50-90% Morinda citrifolia fruit juice by weight, and 0.1-30% non-Morinda citrifolia based fruit juices by weight.

28. A method as in claim 22, additionally comprising 0.1-50% water by weight.

29. A method as in claim 22, wherein one fluid ounce or more is administer to the patient daily.

30. A method as in claim 22, wherein at least three fluid ounces are administered to the patient daily.

31. A method for chemically preventing and inhibiting carcinogenesis at the initiation stage by chemically blocking carcinogens, scavenging free radicals, quenching lipid peroxides, and inhibiting COX-2, said method comprising the steps of:
administering a nutraceutical composition to a patient, wherein said nutraceutical composition comprises processed Morinda citrifolia product or extract present in an amount between about 0.001 and 100 percent by weight, and methylsulfonylmethane present in an amount between about 0.001 and 99 percent by weight.

32. A method as in claim 31, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

33. A method as in claim 31, wherein said Morinda citrifolia product is used with a carrier medium.

34. A method as in claim 31, wherein said composition is administered by process comprising one or more of the following methods: orally, transdermally, injection, intravenously, topically or administered systemically.

35. A method as in claim 31, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

36. A method as in claim 31, wherein said composition is comprised of 0.1-49.9% methylsulfonylmethane by weight, 50-90% Morinda citrifolia fruit juice by weight, and 0.1-30% non-Morinda citrifolia based fruit juices by weight.

37. A method as in claim 31, additionally comprising 0.1-50% water by weight.

38. A method as in claim 31, wherein one fluid ounce or more is administer to the patient daily.

39. A method as in claim 31, wherein at least three fluid ounces are administered to the patient daily.

40. A method for chemically preventing and inhibiting carcinogenesis at the initiation stage by chemically blocking carcinogens, scavenging free radicals, quenching lipid peroxides, and inhibiting COX-2, said method comprising the steps of:
administering a nutraceutical composition to a patient, wherein said nutraceutical composition comprises processed Morinda citrifolia product or extract present in an amount between about 0.001 and 100 percent by weight, and methylsulfonylmethane present in an amount between about 0.001 and 99 percent by weight.

41. A method as in claim 40, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate, extracted elements from Morinda citrifolia.

42. A method as in claim 40, wherein said Morinda citrifolia product is used with a carrier medium.

43. A method as in claim 40, wherein said composition is administered by process comprising one or more of the following methods: orally, transdermally, injection, intravenously, topically or administered systemically.

44. A method as in claim 40, wherein said composition is comprised of 0.1-49.9% methylsulfonylmethane by weight, 50-90% Morinda citrifolia fruit juice by weight, 0.1-50% water by weight and 0.1-30% non-Morinda citrifolia based fruit juices by weight.

45. A method as in claim 40, wherein said composition is comprised of 0.1-49.9% methylsulfonylmethane by weight, 50-90% Morinda citrifolia fruit juice by weight, and 0.1-30% non-Morinda citrifolia based fruit juices by weight.

46. A method as in claim 40, additionally comprising 0.1-50% water by weight.

47. A method as in claim 40, wherein one fluid ounce or more is administer to the patient daily.

48. A method as in claim 40, wherein at least three fluid ounces are administered to the patient daily.

49. A method for preventing, inhibiting and treating breast cancer, comprising the steps of:
adding one or more processed Morinda citrifolia products to an alcohol-based solution;
isolating and extracting an active ingredient of Morinda citrifolia from said solution;
combining said extracted active ingredient with about 100-2000 miligrams of methylsulfonylmethane to form a nutraceutical composition
exposing said nutraceutical composition to an area afflicted by one or more carcinogenic cells, wherein said extracted active ingredient inhibits, prevents, and destroys the growth of said carcinogenic cells.

50. A method as in claim 49, wherein said processed Morinda citrifolia product is comprised of one or more of the following: Morinda citrifolia fruit juice, Morinda citrifolia oil extract, Morinda citrifolia dietary fiber, Morinda citrifolia puree juice, Morinda citrifolia puree, Morinda citrifolia fruit juice concentrate, Morinda citrifolia puree juice concentrate.

51. A method as in claim 49, wherein said nutraceutical composition is used with a carrier medium.

52. A method as in claim 49, wherein said composition is administered by process comprising one or more of the following methods: orally, transdermally, injection, intravenously, topically or administered systemically.

53. A method as in claim 49, wherein said composition is comprised of about 100-2000 milligrams of methylsulfonylmethane, 50-90% Morinda citrifolia fruit juice by weight, 0.1-50% water by weight and 0.1-30% non-Morinda citrifolia based fruit juices by weight.

54. A method as in claim 49, wherein said composition is comprised of about 100-2000 milligrams methylsulfonylmethane by weight, 50-90% Morinda citrifolia fruit juice by weight, and 0.1-30% non-Morinda citrifolia based fruit juices by weight.

55. A method as in claim 49, additionally comprising 0.1-50% water by weight.

56. A method as in claim 49, wherein one fluid ounce or more is administer to the patient daily.

57. A method as in claim 49, wherein at least three fluid ounces are administered to the patient daily.

58. The method of claim 49, wherein said alcohol-based solution is selected from the group consisting essentially of methanol, ethanol, and ethyl acetate, and other alcohol-based derivatives.

59. A method for inhibiting, preventing, and destroying carcinogenic cells within the mammary region of the breast, said method comprising the steps of:
orally administering at least one ounce of a food product comprising processed Morinda citrifolia fruit juice and methylsulfonylmethane on an empty stomach in the morning; and
orally administering at least one ounce of said food product prior to sleeping at night.
